(19) United States
(12) Patent Application Publication
(10) Pub. No.: US 2014/0135798 A1
(43) Pub. Date: May 15, 2014

(54) LIGHT THERAPY SKIN DEVICE

(71) Applicant: Jonathan David, Hermosa Beach, CA (US)

(72) Inventor: Jonathan David, Hermosa Beach, CA (US)

(21) Appl. No.: 14/079,480

(22) Filed: Nov. 13, 2013

Related U.S. Application Data
(60) Provisional application No. 61/725,935, filed on Nov. 13, 2012.

(51) Int. Cl. A61B 17/3205

(52) U.S. Cl. CPC A61B 17/3205
USPC 606/131

(57) ABSTRACT

The present invention is directed to devices for cleansing, beautifying, and improving the health of skin through exfoliation, brushing, and light therapy. A light therapy device may include a detachable treatment head having a treatment surface. One or more light sources may be located behind the detachable treatment head causing specific wavelengths of light to be emitted through detachable treatment head and/or treatment surface. For example if the treatment surface is comprised of bristles forming a brush, the light may travel inside or along the surface of the bristles. One or more buttons located on the device may be used to activate the light source and select a particular wavelength, such as such as 420 nm for blue or 550 nm for red. A switch may also be used to control the fluency or amount of light energy delivered to the skin.

### CLAIM OF PRIORITY UNDER 35 U.S.C. §119

The present Application for Patent claims priority to U.S. Provisional Application No. 61/725,935 entitled “Light Therapy Skin Brush”, filed Nov. 13, 2012, and hereby expressly incorporated by reference herein.

### FIELD

Various features relate to skin treatment and therapy, and more particularly to a devices, system, and accessories for cleansing, beautifying, and improving the health of skin through exfoliation, brushing, and light therapy.

### BACKGROUND

Motorized skin brushes and pads are used to exfoliate and clean the appearance of the skin surface. Typically, a device generates a vibration, oscillation, or rotation to a treatment end pad or brush that comes in contact with the skin, and any topical ingredient on the skin, to wipe away or slough off skin surface products, such as sunscreens, cream and environmental factors as well as gentle removal of the dead skin cells. This helps create a smoother, more reflective skin surface that looks healthy and feels better to the touch.

Light therapy used for dermatological purposes exposes the skin to specific wavelengths of light using lasers, light emitting diodes, florescent lamps, dichroic lamps or very bright full spectrum light, usually controlled by devices. This light is administered for specific periods of time and intensity to the treatment surface to aid in the improvement of photo aging, acne, age related wrinkling of the skin. In light emitting dermatological devices, goggles or closing of the eyes are recommended to prevent damage to the vision of users.

Ultra-violet (UV) light has been used across many applications to sterilize medical and personal tools. One such example is the use of UV lamps to sterilize tooth brushes.

Additionally, fans and other mechanisms of moving air can quickly dry surfaces, thus mitigating the growth of certain microbial, such as bacteria and germs. Currently, there are no motorized or un-motorized skin brushes or pads that combine cleansing and exfoliating light therapy as well as a mechanism to sterilize the brush extending its useful life.

Consequently, a light therapy skin device that combines cleansing, exfoliating light therapy as well as a mechanism for sterilizing the brush is needed.

### SUMMARY

The following presents a simplified summary of one or more implementations in order to provide a basic understanding of some implementations. This summary is not an extensive overview of all contemplated implementations, and is intended to neither identify key or critical elements of all implementations nor delineate the scope of any or all implementations. Its sole purpose is to present some concepts of one or more implementations in a simplified form as a prelude to the more detailed description that is presented later.

According to one aspect, a skin treatment device is provided. The skin treatment device includes a handle portion for gripping the device; and a head portion integrally connected to and extending outwardly from the handle portion. The head portion includes a motion generating mechanism having a shaft extending outwardly; a detachable treatment head having a treatment surface for treating skin of a user and a tubular receiving portion adapted to receive the shaft of the motion generating mechanism; and one or more light sources located behind the detachable treatment head, the one or more light sources emit light through the detachable treatment head for sterilizing the treatment surface.

The device may further include a proximity sensor located on the handle portion below the detachable treatment head. The proximity sensor is adapted to activate the emission of light from the one or more light sources when the treatment surface of the detachable treatment head is located within a pre-determined distance from the skin of the user.

According to one feature, the treatment surface is a brush having bristles extending outwardly therefrom. The bristles may be arranged in circular rows. A first outer row in the circular rows may remain stationary while inner rows of the circular rows move (e.g. rotate).

According to another aspect, a skin treatment device adapted to be received within a charging base is provided. The skin treatment device includes a handle portion for gripping the device; and a head portion integrally connected to and extending outwardly from the handle portion. The head portion includes a motion generating mechanism, the motion generating mechanism having a shaft extending outwardly; a detachable treatment head having a treatment surface for treating skin of a user and a tubular receiving portion adapted to receive the shaft of the motion generating mechanism; and one or more light sources located behind the detachable treatment head, the one or more light sources emit light through the detachable treatment head for sterilizing the treatment surface.

The charging base includes a lower portion and an upper portion where the upper portion is integrally connected to and extends upwardly from the lower portion. The lower portion of the base is adapted to receive a bottom of the handle portion and the upper portion of the base is adapted to receive the detachable treatment head. The one or more base light sources are located within the upper portion of the charging base and adapted to emit light to the treatment surface when placed in the upper portion for sterilizing the treatment surface.

According to one feature, the charging base has a clear or opaque cover located over the base light sources for preventing the treatment surface from coming into direct contact with the base light sources.

According to one feature, the charging base further comprises a drying mechanism, such as a fan, located in the upper portion of the charging base between the cover and the base light source for drying the treatment surface.

### BRIEF DESCRIPTION OF THE DRAWINGS

FIG. 1 illustrates a perspective view of a skin treatment device in a base, according to one embodiment.

FIG. 2 illustrates a perspective view of the skin treatment device of FIG. 1 separated from the base and the brush cover removed.

FIG. 3 illustrates an exploded perspective view of the skin treatment device of FIG. 1.

FIG. 4 illustrates a side elevation view of the skin treatment device of FIG. 1.

FIG. 5 illustrates a front elevation view of the skin treatment device of FIG. 1.

FIG. 6 illustrates a cross sectional view of the skin treatment device and base of FIG. 1.

FIG. 7 illustrates a fragmentary view of the head portion of a skin treatment device, according to one embodiment.

FIG. 8 illustrates an application of the skin treatment device of FIG. 1 on facial skin of a user.

FIG. 9 illustrates a side elevation view of the skin treatment device of FIG. 1 in a base for sterilizing and drying the treatment surface of the skin treatment device, according to one embodiment.

### DETAILED DESCRIPTION

In the following detailed description, numerous specific details are set forth in order to provide a thorough understanding of the embodiments. However, it will be understood by one of ordinary skill in the art that the embodiments may be practiced without these specific details. For example, operations may be shown in block diagrams, or not be shown at all, in order not to obscure the embodiments in unnecessary detail. In other instances, well-known operations, structures and techniques may not be shown in detail in order not to obscure the embodiments.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Overview

Embodiments of the invention are directed to devices for cleansing, beautifying, and improving the health of skin through exfoliation, brushing, and light therapy. A light therapy device, as described herein, may include a detachable treatment head having a treatment surface (i.e. the surface of the device which comes into contact with the skin of a user). One or more light sources may be located behind the detachable treatment head causing specific wavelengths of light to be emitted out from the detachable treatment head and/or treatment surface. For example if the treatment surface is comprised of bristles forming a brush, the light emitted may travel inside or along the surface of the bristles. One or more switches located on the device may be used to activate the light source and select a particular wavelength, such as such as 420 nm for blue or 550 nm for red. An additional switch may also be used to control the fluency or amount of light energy delivered to the skin. For example, one position of the switch may be 5 J/cm2 while another may be 10 J/cm2.

According to one feature, a microchip located within the device may be utilized to control or program the specific wavelength of light that is transmitted to the skin of a user (or consumer) through the treatment surface of the device. The microchip may be programmed during the manufacturing process to transmit one or more wavelengths (or color) when the device. Alternatively, the consumer or other secondary user could be allowed to program the microchip to release other wavelengths or colors after the device has been purchased. For example, the end user may program the device by pressing a specific sequence(s) of buttons (or switches) on the device. A specific sequence may be associated with a particular wavelength.

According to another feature, the device may be programmed to notify the user to move the treatment surface of the device to a different location on the skin. For example, after a specific time period (e.g. 30 seconds) has elapsed, a tactile, visual or audible notification may be provided to the user.

According to another feature, a proximity sensor located on the handle portion below the detachable treatment head, may be used to activate the emission of light when the treatment surface is within a pre-determined distance of the skin of the user. For example, the light would not be emitted through the treatment surface unless the treatment surface was within one inch of the skin of the user. The pre-determined distance may be programmed during the manufacturing process or the consumer may be able to program the pre-determined distance.

According to another feature, the device may also be used in combination with photo activated topical ingredients to improve or accelerate the desired effect on the skin.

According to another feature, the device may also make use of a UV light source either in the handle portion or the head portion, or as part of an external accessory, to sterilize the treatment surface of the device. For example, a charging base may also have a sterilizing port, whereby the treatment surface of the device is placed in the port or opening of the charging base and subsequently sterilized by a UV light source emitted onto the treatment surface of the device.

According to another feature, air provided through or along the treatment surface of the device may be utilized to accelerate the drying of the treatment surface of the device to prevent bacteria and mold from growing. The air may be emitted from the device, such as in the handle portion or the head portion, or from an external device, such as the charging base. For example, a charging base may also have a drying port, whereby the treatment surface is placed in the drying port which contains a source of moving air that helps dry the treatment surface after use.

According to another feature, both the aforementioned UV light sterilizing port and the air drying port can be combined as one port that can work separately or together.

### Light Therapy Device

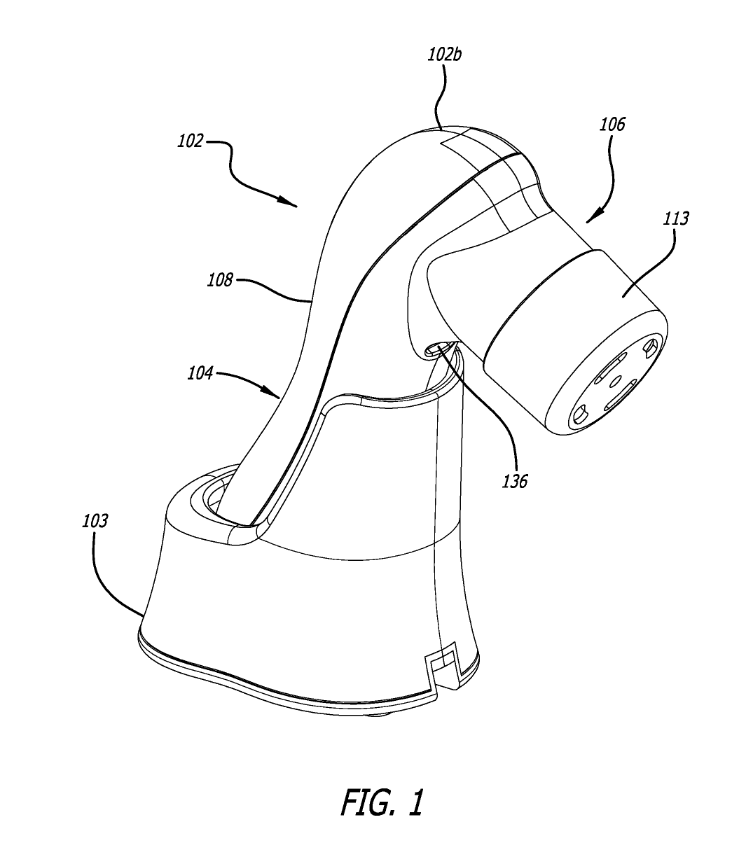

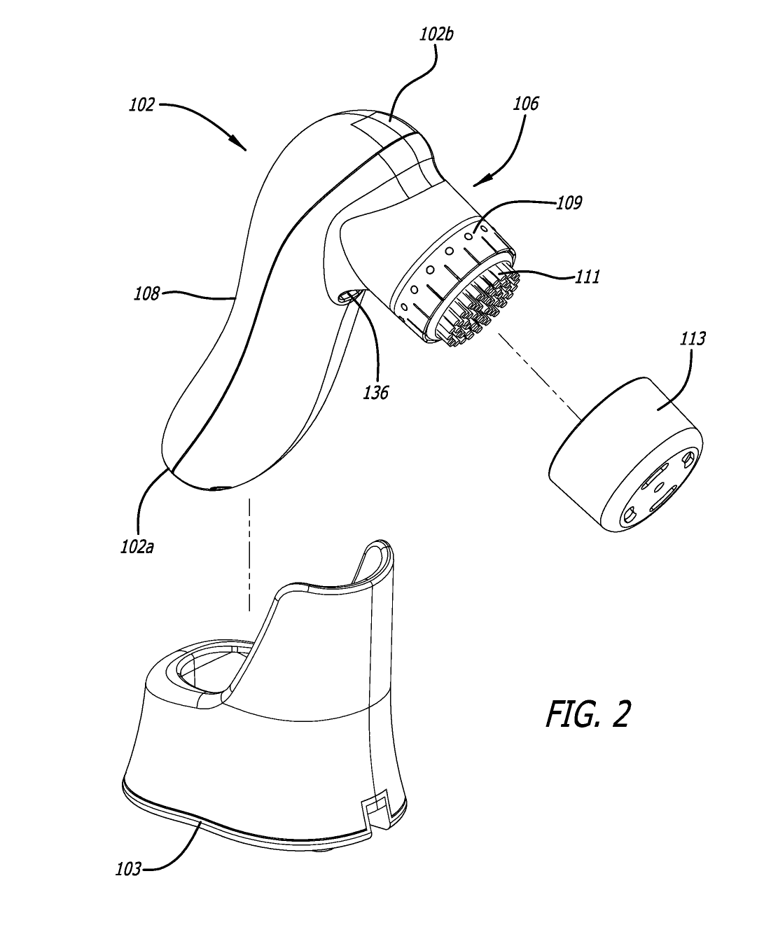

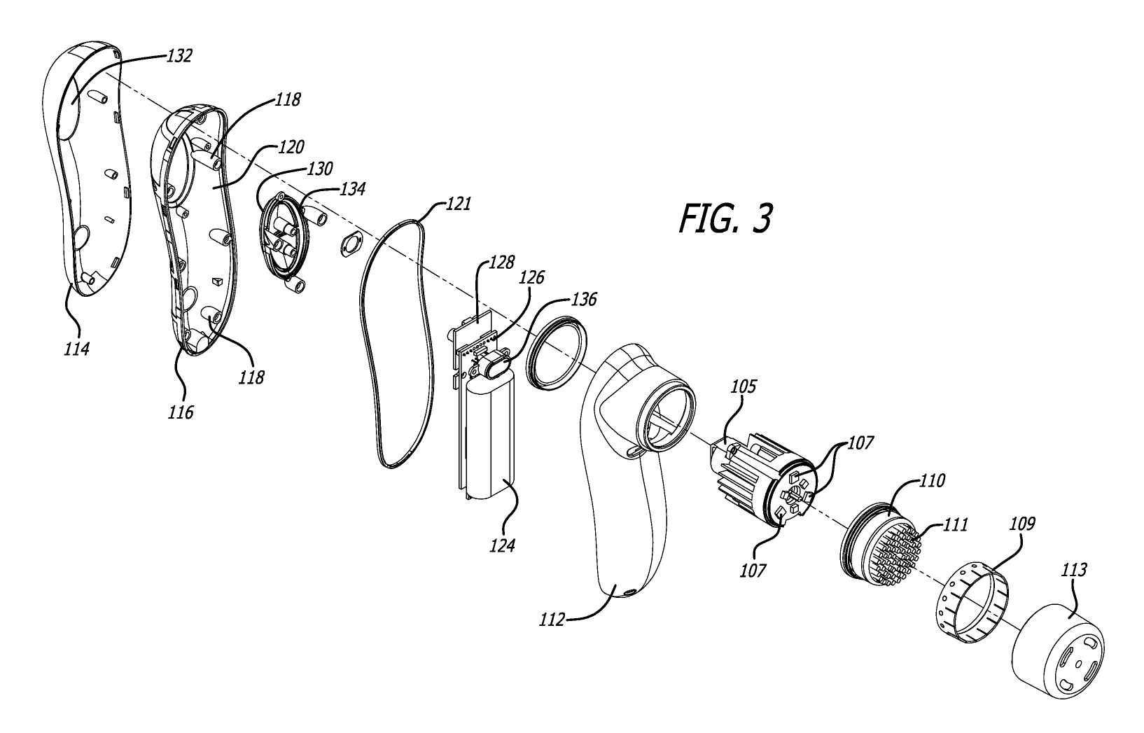

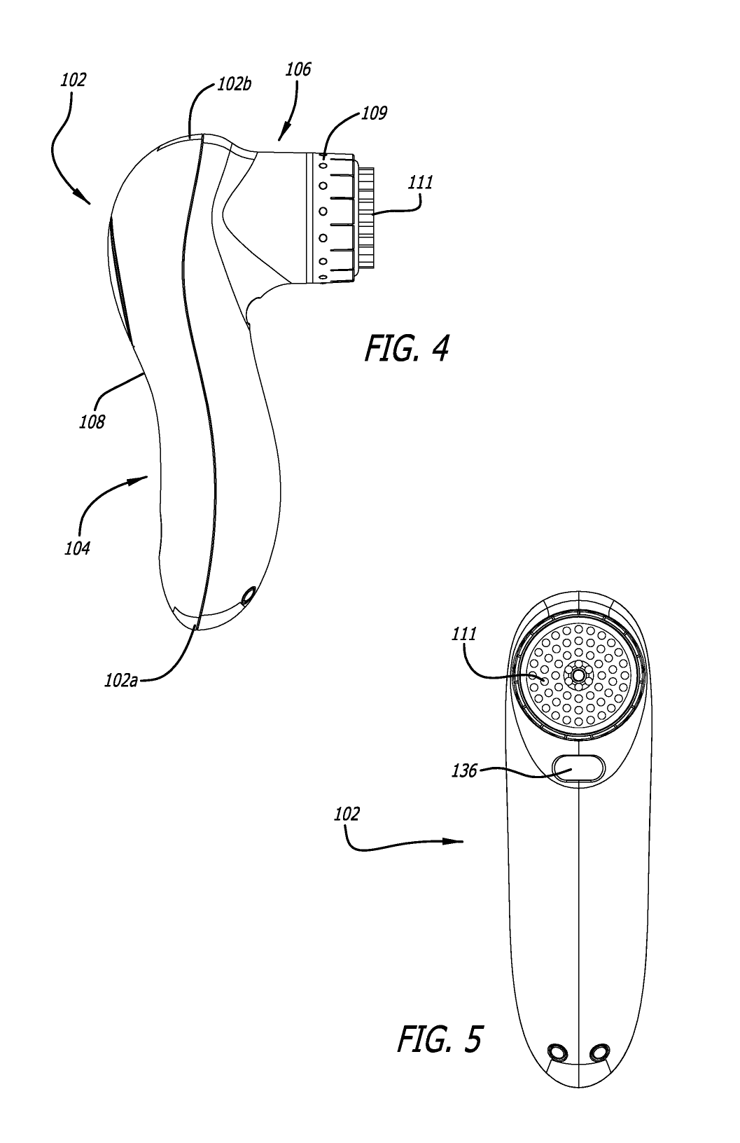

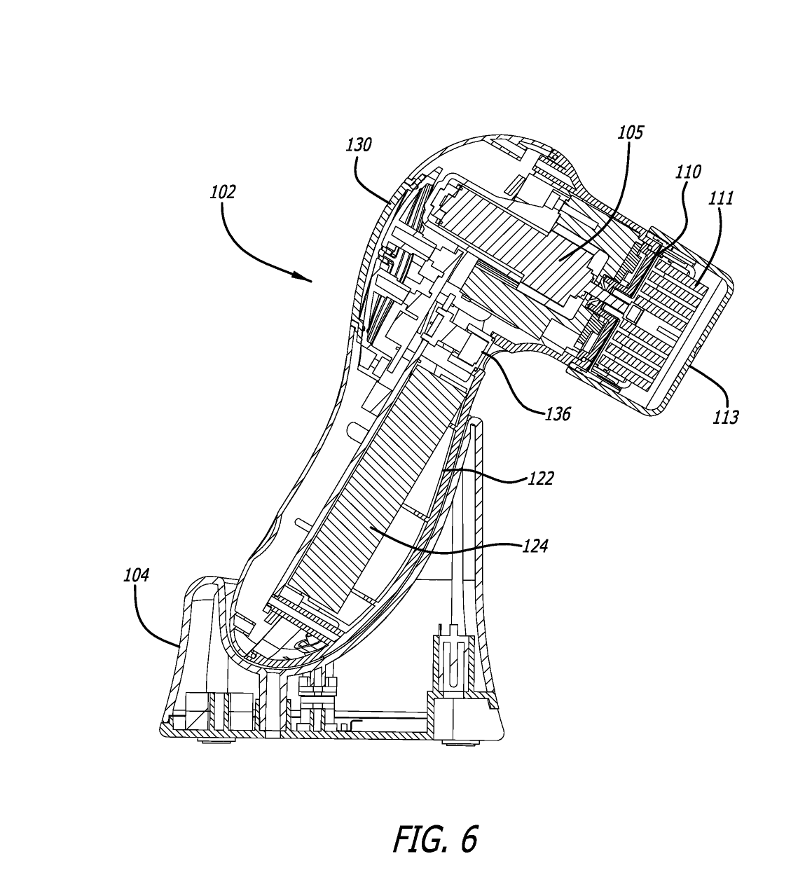

FIG. 1 illustrates a perspective view of a skin treatment device 102 in a base 103, according to one embodiment. FIG. 2 illustrates a perspective view of the skin treatment brush of FIG. 1 separated from the base and a treatment head cover removed. FIG. 3 illustrates an exploded perspective view of the skin treatment device of FIG. 1. FIG. 4 illustrates a side elevation view of the skin treatment device of FIG. 1. FIG. 5 illustrates a front elevation view of the skin treatment device of FIG. 1. FIG. 6 illustrates a cross sectional view of the skin treatment device and base of FIG. 1. The following discussion refers interchangeably to FIGS. 1-6.

As shown, the skin treatment device 102 is adapted to be placed into the base 103. The base 103 may be used to charge the skin treatment device 102 as well as provide power to light source(s) on or within the device 102, as described in further detail below.

The device 102 comprises a handheld unit having a handle portion 104 for gripping the device 102 and a head portion 106 comprising a motion generating mechanism 105, one or more light sources 107, such as light emitting diodes that transmit specific wavelengths of light to the skin, and a detachable treatment head 110 having a treatment surface 111. An application of the skin treatment device of FIG. 1 on facial skin of a user is illustrated in FIG. 8.

The handle portion 104 generally extends from a proximal end 102a to a distal end 102b of the device 102, with the head portion 106 extending outwardly from the distal end 102b. The detachable treatment head 110 may include a receiving portion adapted to receive within a shaft from the motion generating mechanism 105 in the head portion 106 for securing the detachable treatment head 110 to the head portion 106 and providing a means of motion for the detachable treatment head 110. The treatment surface 111 may be a brush, as shown, a pad or any other abrasive or cleaning surface. The treatment surface 111 can move across the skin of a user at sonic or ultrasonic speeds. Once powered On, a button on the device may be used to switch the speeds in which the detachable treatment head 110 moves.

To prevent or reduce light emitting out the side of the head portion 106, a light reducing member 109 may be detachably placed over a gap where the detachable head portion 110 is joined to the head portion 106 of the device. The light reducing member 109 may be made from a material such as a non-translucent rubber. To keep the treatment surface 111 clean when not in use, a removable cap 113 may be placed over the treatment surface 11 and secured to the detachable treatment head 110 by a friction fit.

The components of the present device 102 are illustrated in FIGS. 3 and 6. For manufacturing convenience, a housing 108 of the device 102 illustrated in FIGS. 1-9 comprises a front housing portion 112 and a rear housing portion 114 which join to form the housing 108 and enclose the internal components of the device. In the illustrated embodiment, the components contained in the handle portion 104 are retained in the housing 108 by a cover 116, and the cover 116 is joined to the front housing portion 112. A flange 121 may be located between the cover 116 and the front housing portion 112 for ensuring a tight fit.

According to one embodiment, the cover 116 is joined to the front housing portion 112 by fitting one or more shafts 118 extending away from the interior surface 120 of the cover 116 (i.e., inwardly) into the opening of one or more tubular receiving portions extending away from the interior surface 122 of the front housing portion 112 (i.e., inwardly). The receiving portions are sized to receive the shafts 118 and retain them with a friction fit, thereby retaining the front housing portion 112 and the cover 116 together. The shafts 118 can alternatively be retained on the rear housing portion 114, or the receiving portions can alternatively be positioned on the rear housing portion 114 and the shafts 118 on the cover 116 or the front housing portion 112. Other ways to join the front housing portion 112 and the rear housing portion 114 known to the art can also be used. The rear housing portion 114 is placed over the cover 116, and can be retained on the cover 116 by means of an adhesive, a friction fit, or in other ways known to the art. The housing 108 is preferably formed from a rigid material such as a rigid plastic polymer.

Contained within the housing 108 of the handle portion 104 in this embodiment is a battery 124, one or more printed circuit boards 126, an On/Off switch 128 and a proximity sensor 136. The battery 124 is preferably rechargeable and can be charged by placing in the base (or charging station) 104. Alternatively, the battery 124 may be placed in electrical communication with a connector for connection to an electrical cord, in order to place the battery 124 in electrical communication with an electrical outlet or other source of power. The battery 124 provides power to the motion generating mechanism 105, the one or more light sources 107, the one or more printed circuit boards 126 and the proximity sensor 136. A programmable microchip may be located on the one or more printed circuit boards 126 that can be used to select which wavelengths the device can transmit to the skin.

Power to the device 100 can be controlled with the On/Off switch 128 which is preferably actuated by depressing a button 130. The button 130 is retained within an opening 132 of the rear portion 116 of the housing 108, preferably in the distal end 102b of the device 100, and includes a circumferential flange 134 maintained within the interior of the housing 108 in order to secure the button 130 to the device 100. The button 130 is preferably formed from an elastomeric or other flexible material, and actuates the switch 128 when a user applies pressure to the exterior surface of the button 130. When the switch 128 is in the On position, power is supplied to the motion generating mechanism 105 which causes the shaft of the motion generating mechanism 105 to move, such as in a rotational direction, which in turn causes the detachable treatment head 110 and the treatment surface 111 to move along the skin of the user. The motion can be a vibrating, oscillating or rotating motion.

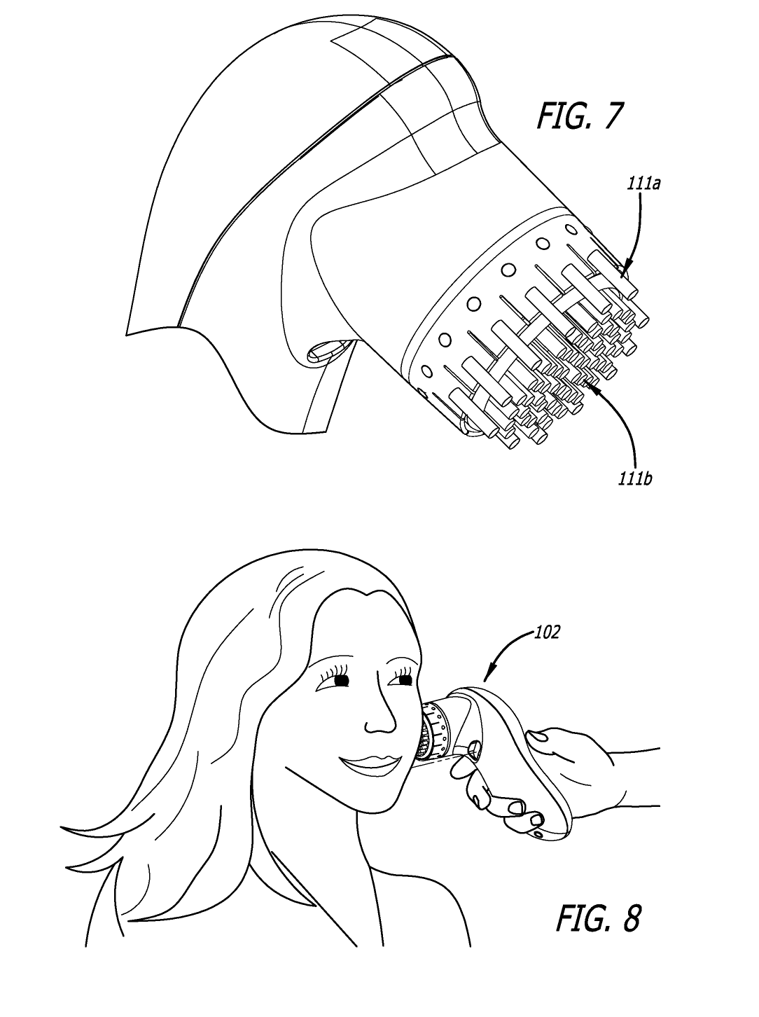

According to one embodiment, the treatment surface 111 of the detachable treatment head 110 may include an outermost circular row of bristles 111a which are stationary and a plurality of inner circular rows of bristles 111b which may rotate. FIG. 7 illustrates a fragmentary view of the treatment surface 111 of the detachable treatment head 110 of a skin treatment device, according to one embodiment.

The button 130 described above may also include a portion for controlling the wavelength specific light sources located in or on the motion generating mechanism 105. A top portion of the button 130 may be used for turning the power On and Off while a bottom portion of the button 130 may be used selecting a specific wavelength of light to be emitted to reach the skin of the user.

### Proximity Sensor

The device may also include a proximity sensor 136 located on the handle portion 104 below the detachable treatment head 110, may be used to activate the emission of light when the treatment surface is within a pre-determined distance of the skin of the user. According to one embodiment, the transmission of light from the light sources 107 occurs only when the skin (or other surface to be treated) is within a pre-determined distance from the proximity sensor 136. The pre-determined distance may be pre-programmed into the device. As a result, eye discomfort is reduced and eye safety is increased.

### UV Cleaning

According to one embodiment, the one or more of the light sources 107 in the head portion 110 may be utilized as a cleaning mechanism and emit wavelengths of light, such as ultra violet (UV) wavelengths of light for reducing microbial, such as bacteria. The emitted light is transmitted through the treatment surface 111 on the detachable treatment head 110 sterilizing the detachable head 100 and the treatment surface 111 killing any microbial. Alternatively, the light sources 107 may be located in the handle portion 106 or as part of an external accessory such as a charging base. The cleaning mechanism may be initiated by depressing a button on the device 100 and/or when the device 100 is placed into its charging base.

The proximity sensor 136 described above may be used to prevent the light sources from emitting potentially damaging wavelengths of light if the sensor 136 is within a pre-determined distance of another surface, such as an eye or the skin of the user. The detachable cap 113 placed over the treatment surface may be used to contain the germicidal light. Affixing the cap 113 may activate a magnetic or other switch preventing the emission of germicidal light when the cap is not affixed. Alternatively, the device may be programmed such that the germicidal light is turn On or Off in timed intervals. For example, when placed in the charging base 103, the germicidal light may be emitted for thirty (30) minutes before shutting Off. The germicidal light may also turn itself On and Off at pre-determined intervals, such as every twelve (12) hours.

### Treatment Surface Having Stationary and Moving Bristles

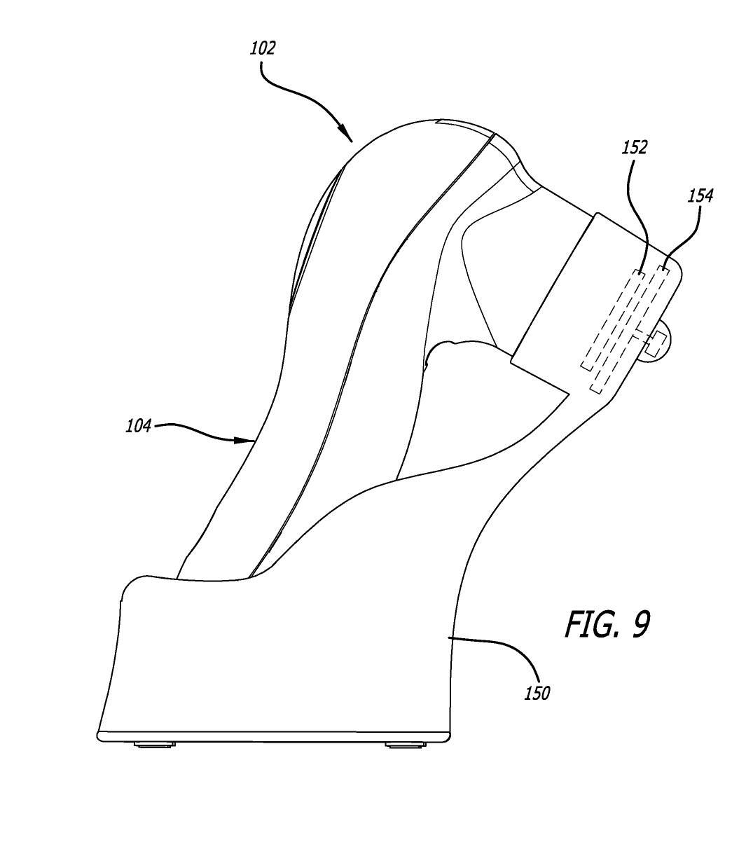

FIG. 9 illustrates a side elevation view of the skin treatment device of FIG. 1 in a base for sterilizing and drying the treatment surface 111 of the skin treatment device 102, according to one embodiment. To sterilize and dry the treatment surface 111 of the detachable treatment head 110, the device may be placed into a base 150 as shown in FIG. 9. The base 150 may include a lower portion and an upper portion, the upper portion integrally connected to and extending upwardly from the lower portion. A lower opening or port in the lower portion is adapted to receive a bottom end of the handle portion 104 and an upper opening or port in the upper portion is adapted to receive the detachable treatment head 110. When the device is placed in the base 150 and the base 150 is plugged into an electrical outlet the battery 124 is charged. Additionally, placing the device in the base 150 may cause a cleaning or light source 152, located in the base 150, to transmit a sanitizing wavelength of light, such as UV light, to the treatment surface 111 on the detachable treatment head 110. The light emanating from the light source 152 may pass through a clear or opaque window that keeps the treatment surface 111 from coming into contact with the light source 152. The light source 152 may be connected to a sensing switch which causes the light source 152 to be turned On when the device is placed in the base 150. Additionally, a built in timing mechanism may be used to limit the amount of time the light source 152 is transmitting light. The base 150 may also include an air source 154, or drying mechanism for moving air. The air source 124 may be a fan or other mechanism that that causes air to be moved in and around the treatment surface 111 of the device 102. The air source 157 may be in addition to or instead of the light source 152. According to one embodiment, the drying mechanism 124 is located in the upper portion of the charging base 150 and activated when the device is placed in the charging base 102 causing air to be blown on the treatment surface 111.

Those of skill in the art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

1. A skin treatment device, comprising:
a handle portion for gripping the device; and
a head portion integrally connected to and extending outwardly from the handle portion, the head portion comprising:
a motion generating mechanism, the motion generating mechanism having a shaft extending outwardly;
a detachable treatment head having a treatment surface for treating skin of a user and a tubular receiving portion adapted to receive the shaft of the motion generating mechanism; and
one or more light sources located behind the detachable treatment head, the one or more light sources emit light through the detachable treatment head for sterilizing the treatment surface.

2. The device of claim 1, further comprising a proximity sensor located on the handle portion below the detachable treatment head, the proximity sensor adapted to activate the emission of light from the one or more light sources when the treatment surface of the detachable treatment head is located within a pre-determined distance from the skin of the user.

3. The device of claim 1, wherein the treatment surface is a brush having bristles extending outwardly therefrom.

4. The device of claim 1, wherein the bristles are arranged in circular rows.

5. The device of claim 4, wherein a first outer row in the circular rows remains stationary while inner rows of the circular rows move.

6. The device of claim 1, wherein the motion generating mechanism causes the treatment surface to have a vibrational, oscillation, or rotational motion.

7. The device of claim 1, wherein the treatment surface is an abrasive pad.

8. A skin treatment device adapted to be received within a charging base, comprising:
a handle portion for gripping the device; and
a head portion integrally connected to and extending outwardly from the handle portion, the head portion comprising:
a motion generating mechanism, the motion generating mechanism having a shaft extending outwardly;
a detachable treatment head having a treatment surface for treating skin of a user and a tubular receiving portion adapted to receive the shaft of the motion generating mechanism; and
one or more light sources located behind the detachable treatment head, the one or more light sources emit light through the detachable treatment head for sterilizing the treatment surface; and
wherein the charging base includes a lower portion and an upper portion, the upper portion integrally connected to and extending upwardly from the lower portion;
wherein the lower portion of the base is adapted to receive a bottom of the handle portion and the upper portion of the base is adapted to receive the detachable treatment head;
wherein one or more base light sources are located within the upper portion of the charging base and adapted to emit light to the treatment surface when placed in the upper portion for sterilizing the treatment surface.

9. The device and charging base of claim 8, further comprising:
a clear or opaque cover located over the base light sources for preventing the treatment surface from coming into direct contact with the base light sources.

10. The device and charging base of claim 9, further comprising:
a drying mechanism located in the upper portion of the charging base between the cover and the base light source for drying the treatment surface.

11. The device of claim 10, wherein the drying mechanism in a fan.

12. The device of claim 8, further comprising:
a proximity sensor located on the handle portion below the detachable treatment head and adapted to activate the emission of light from the one or more light sources when the treatment surface of the detachable treatment head is located within a pre-determined distance from the skin of the user.

13. The device of claim 8, wherein the treatment surface is a brush having bristles extending outwardly therefrom.

14. The device of claim 8, wherein the bristles are arranged in circular rows.

15. The device of claim 14, wherein a first outer row in the circular rows remains stationary while inner rows of the circular rows move.

16. The device of claim 8, wherein the motion generating mechanism causes the treatment surface to have a vibrational, oscillation, or rotational motion.

17. The device of claim 8, wherein the treatment surface is an abrasive pad.
